(19) United States
(12) Patent Application Publication
(10) Pub. No.: US 2020/0324010 A1
(43) Pub. Date: Oct. 15, 2020

(54) Negative Air Discharge Apparatus

(71) Applicant: Stephen F.H. Gibbs, Glencoe (CA)

(72) Inventor: Stephen F.H. Gibbs, Glencoe (CA)

(21) Appl. No.: 16/383,777

(22) Filed: Apr. 15, 2019

(51) Int. Cl. A61L 9/00; F24F 3/16

(52) U.S. Cl. CPC A61L 9/00; A61M 2205/3331; F24F 3/161

(57) ABSTRACT

A negative air discharge apparatus configured to be operably coupled to a negative air machine so as to facilitate the discharge of air from the negative air machine into the interior of a building. The negative air discharge apparatus includes a housing having a plurality of walls, a top and a bottom configured to form an interior volume. The housing has operably coupled thereto an air intake wherein the air intake is configured to be coupled to a negative air machine via a flexible air duct. The negative air discharge apparatus further includes an air discharge port proximate the bottom of the housing that is operable to discharge air from the negative air machine. The housing further includes a first access and a second access that are configured to provide entry into the interior volume of the housing and the plurality of air filters disposed therein.

### FIELD OF THE INVENTION

The present invention relates generally to negative air control techniques, more specifically but not by way of limitation, a negative air discharge apparatus wherein the present invention is operably coupled to a negative air machine wherein the present invention provides contamination control so as to discharge the air within a facility as opposed to outside the facility.

### BACKGROUND

As is known in the art, negative air machines are utilized for various purposes such as but not limited to construction sites within environments like hospitals. Negative air machines remove potentially dangerous particles from the air by creating a negative air pressure within a desired space of a facility. The negative air machines essentially create a vacuum within an area and remove the air therefrom and transfer the air most often to outside of the building. As the air is removed it is sometimes filtered and the air is exhausted outside of the building through a flexible hose or similar element. Negative air machines assist in the reduction of the spread of pollutants to other areas inside the building. Negative air machines are often utilized during construction in hospitals wherein the construction area within a hospital must be maintained at a negative pressure and the air transferred to outside of the building.

While the conventional negative air machine is configured to vent air outside of the building, there can be construction areas within a hospital that are located in a portion of the building wherein the negative air discharge cannot be routed to outside of the building. As a result, this presents a challenge for an area that requires negative air pressure during the construction that is taking place.

Accordingly, there is a need for a negative air discharge apparatus that is configured to be operably coupled to a negative air machine wherein the negative air discharge apparatus provides filtration of the air from the negative air discharge machine and discharges the air within an area in the facility adjacent to the required negative air area.

### SUMMARY OF THE INVENTION

It is the object of the present invention to provide a negative air discharge apparatus that is configured to be operably coupled to a negative air machine wherein the present invention provides the ability to discharge the air from the negative air machine within the facility.

Another object of the present invention is to provide a negative air discharge apparatus that provides additional filtration of contaminants from the air introduced thereinto wherein the present invention includes a housing that is manufactured from a rigid material.

A further object of the present invention is to provide a negative air discharge apparatus that is configured to be operably coupled to a negative air machine wherein the housing includes an intake vent configured to be operable coupled to a flexible air duct hose.

Still another object of the present invention is to provide a negative air discharge apparatus that provides additional filtration of contaminants from the air introduced thereinto wherein the housing a first filter disposed within the interior volume thereof.

An additional object of the present invention is to provide a negative air discharge apparatus that is configured to be operably coupled to a negative air machine wherein the present invention includes a gate valve proximate the bottom of the housing that is operable to direct the air exiting therefrom.

Yet a further object of the present invention is to provide a negative air discharge apparatus that provides additional filtration of contaminants from the air introduced thereinto that further includes a second filter disposed within the interior volume of the housing.

Another object of the present invention is to a provide a negative air discharge apparatus that is configured to be operably coupled to a negative air machine wherein the housing is coated with an anti-microbial material.

An alternate object of the present invention is to provide a negative air discharge apparatus that provides additional filtration of contaminants from the air introduced thereinto wherein the present invention further includes an anemometer to measure and display the air pressure.

Still a further object of the present invention is to provide a negative air discharge apparatus that is configured to be operably coupled to a negative air machine wherein the intake vent of the housing is configured to be operably coupled to a twelve inch flexible air duct hose.

An additional object of the present invention is to provide a negative air discharge apparatus that provides additional filtration of contaminants from the air introduced thereinto that further includes at least one access door operable to provide access to the interior volume of the housing and the filters disposed therein.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is front view of an embodiment of the present invention; and

FIG. 2 is a perspective view of an embodiment of the present invention; and

FIG. 3 is a diagrammatic view of a layout of the present invention in use operably coupled to a negative air machine.

### DETAILED DESCRIPTION

Referring now to the drawings submitted herewith, wherein various elements depicted therein are not necessarily drawn to scale and wherein through the views and figures like elements are referenced with identical reference numerals, there is illustrated a negative air discharge apparatus 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Now referring in particular to the Figures submitted herewith, the negative air discharge apparatus 100 includes a housing 10 manufactured from a lightweight rigid material such as but not limited to aluminum. The housing 10 is formed utilizing a plurality of walls 12, a top 14 and a bottom 14 creating an interior volume. The aforementioned elements are operably coupled utilizing suitable durable techniques such as but not limited to welding. While the embodiments of the negative air discharge apparatus 100 are illustrated herein having four walls 12, it is contemplated within the scope of the present invention that the housing 10 could be formed utilizing various quantities of walls 12 in order to create the necessary interior volume suitable for the desired application of receiving a plurality of air filters therein. It is further contemplated within the scope of the present invention that the housing 10 could have an antimicrobial material disposed on the outer surface thereof so as to facilitate placement of the negative air discharge apparatus 100 in sterile environments.

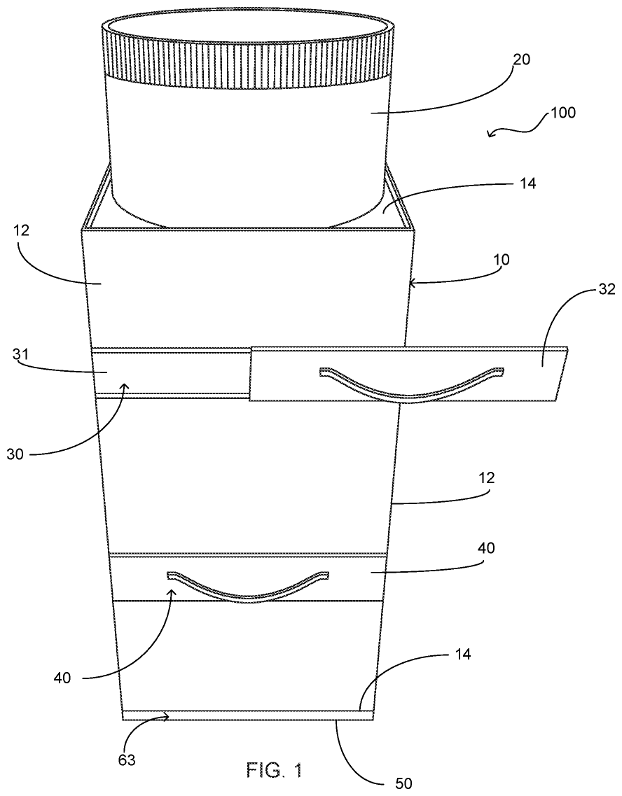

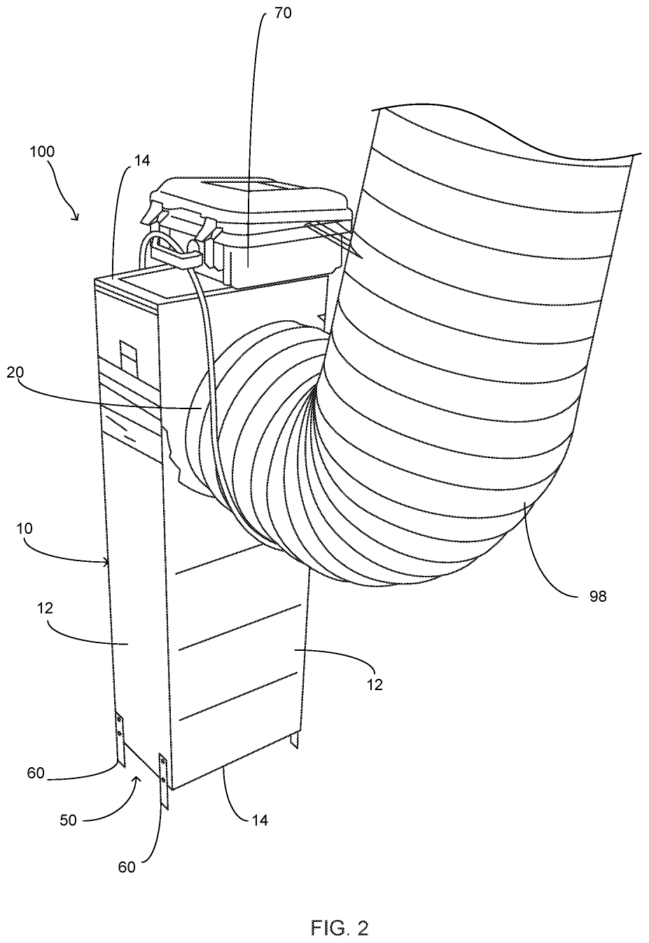

The housing 10 further includes air intake 20 operably coupled thereto. The air intake 20 is formed from a lightweight rigid material and is secured to the housing 10 utilizing suitable durable techniques. As illustrated in the embodiments of FIG. 1 and FIG. 2, the air intake 20 can be positioned either on the top 14 of the housing 10 or on a wall 12. The air intake 20 is annular in shape and in a preferred embodiment is approximately twelve inches in diameter. The air intake 20 is atmospherically coupled to vent hose 98. Vent hose 98 is a conventional flexible air duct hose that is operably coupled to an exemplary negative air machine 99. While the housing 10 is illustrated having one air intake 20, it is contemplated within the scope of the conversation that the housing 10 could have formed thereon more than one air intake vent 20 and wherein the air intake vent 20 could be configured to operably couple with different diameter air duct hoses.

Referring in particular to the embodiment of the negative air discharge apparatus 100 illustrated in FIG. 1, the front wall 13 includes a first access 30 and a second access 40 formed therein. The first access 30 includes an opening 31 that is operably covered by movable door 32 wherein the door 32 is transitioned from a closed position to an open position in order to provide entry into opening 31. It is contemplated within the scope of the present invention that the door 32 could be slidably movable intermediate its closed position and open position. It should however be understood within the scope of the present invention that the door 32 could be movable in alternate manners. The door 32 is moved to its open position in order to gain access to a first air filter (not illustrated herein). The negative air discharge apparatus 100 provides additional filtration of the air being received from the exemplary negative air machine 99 so as to permit the discharge of the air within the interior of a building. It is contemplated within the scope of the present invention that the first air filter could be various types of air filters designed to remove different types of particles and contaminants.

The second access 40 includes door 43 and is illustrated herein in its closed position. The second access 40 provides entry into the interior volume of the housing 10 in order to engage with the second air filter (not illustrated herein). The negative air discharge apparatus 100 in a preferred embodiment includes a first air filter and a second air filter disposed within the interior volume of the housing 10. It is contemplated within the scope of the present invention that the first air filter and second air filter could be various types of air filters configured to remove airborne pathogens and other contaminants. While the preferred embodiment of the negative air discharge apparatus 100 utilizes two air filters, it is contemplated within the scope of the present invention that the negative air discharge apparatus 100 could have alternate quantities of air filters disposed in the interior volume thereof.

The housing 10 includes air discharge port 50 proximate the bottom 14. The air discharge port 50 is configured to have the air entering the air intake 20 to egress from the interior volume of the housing 10 subsequent passing through the air filters. The air discharge port 50 is created in one embodiment through utilizing support stands 60 that are mounted to the housing 10 and configured to elevate the bottom 14 off of the floor on which the negative air discharge apparatus 100 is superposed. The embodiment illustrated in FIG. 1 herein of the negative air discharge apparatus 100 illustrated an alternate style of creating the air discharge port 50 wherein the air discharge port 50 is formed utilizing the walls 12 to create opening 63 that functions to permit air to egress from the interior volume of the housing 10. It is contemplated within the scope of the present invention that the air discharge port 50 could be configured in alternate manners and still achieve the desired objective described herein.

While not illustrated herein, it is contemplated within the scope of the present invention that a deflector or gate valve could be disposed within the interior volume of the housing 10 wherein the deflector is configured to provide directional control of the air exiting the air discharge port 50.

An air pressure measuring unit 70 is disposed on the top 14 of the housing 10 in the embodiment of the negative air discharge apparatus 100. The air pressure measuring unit 70 utilizes the necessary electronics to measure air velocity and pressure to ensure the desired airflow is occurring. The air pressure measuring unit 70 is a conventional instrument commercially available for the described tasks herein.

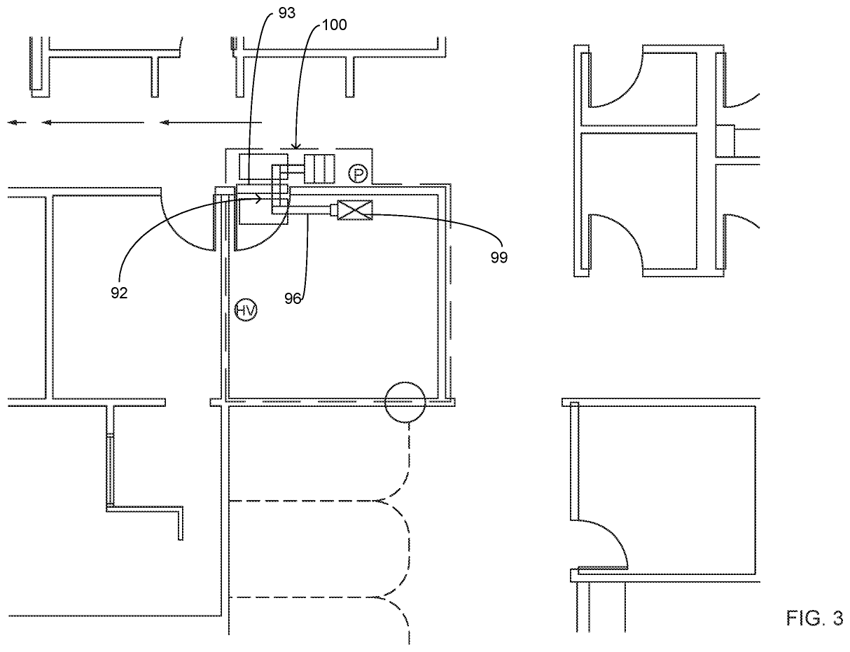

Referring now to FIG. 3 herein, a diagrammatic layout of the utilization of the negative air discharge apparatus 100 is illustrated therein. The negative air discharge apparatus 100 is operably coupled to an exemplary negative air machine 99 that is disposed within an interior room of a building. The negative air machine 99 has an air duct 96 coupled to the negative air discharge apparatus 100 wherein the negative air discharge apparatus 100 is configured to receive and discharge the air therefrom. The air duct 96 is hermetically journaled through a seal 93 that has been installed over the entryway 92 into the room. The aforementioned atmospherically isolates the negative air machine 99 and the negative air discharge apparatus 100 in order to achieve the desired objective as previously described herein where air is filtered and then discharged into the interior of a building utilizing the negative air discharge apparatus 100.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding description is, therefore, not intended to be limited to the specific forms set forth herein, but on the contrary, it is intended to cover such alternatives, modifications, and equivalents, as can be reasonably included within the spirit and scope of the invention.

What is claimed is:

1. A negative air discharge apparatus configured to be operably coupled to a negative air machine and facilitate the discharge of negative air within the interior of a building comprising:
a housing, said housing having at least one wall, a bottom and a top forming an interior volume, said housing being manufactured from a lightweight rigid material, said at least one wall having an outer surface, said housing having an upper end and a lower end;
an air intake, said air intake being operably coupled to said housing, said air intake being proximate said upper end of said housing, said air intake being configured to be operably coupled to an air duct hose, wherein the air duct hose atmospherically couples the negative air discharge apparatus with the negative air machine;
at least one air filter, said at least one air filter being disposed in the interior volume of said housing; and
an air discharge port, said air discharge port being proximate said lower end of housing, said air discharge port configured to direct air outwards from said housing that has entered thereinto via said air intake.

2. The negative air discharge apparatus as recited in claim 1, and further including a first access, said first access being formed in said at least one wall, said first access having an opening, said first access providing entry into the interior volume of said housing via said opening thereof.

3. The negative air discharge apparatus as recited in claim 2, and further including an air deflector, said air deflector being disposed within said interior volume of said housing proximate said air discharge port, said air deflector configured to direct air exiting from the air discharge port.

4. The negative air discharge apparatus as recited in claim 3, wherein said air intake is annular in shape and has a diameter of approximately twelve inches.

5. The negative air discharge apparatus as recited in claim 4, wherein the housing has an antimicrobial material disposed on the outer surface of the at least one wall.

6. The negative air discharge apparatus as recited in claim 5, and further including a pressure measuring unit, said pressure measuring unit operably coupled to said housing, said pressure measuring unit configured to measure and display the air pressure within the housing of the negative air discharge apparatus.

7. A negative air discharge apparatus configured to be operably coupled to a negative air machine and facilitate the discharge of negative air within the interior of a building comprising:
a housing, said housing having a plurality of walls, a bottom and a top forming an interior volume, said housing being manufactured from a lightweight rigid material, said plurality of walls having an outer surface, said housing having an upper end and a lower end;
a first access, said first access being formed in one of said plurality of walls, said first access having an opening, said first access providing entry into the interior volume of said housing via said opening thereof, said first access having a door, said door being movable intermediate a closed position and an open position;
an air intake, said air intake being operably coupled to said housing, said air intake being proximate said upper end of said housing, said air intake being configured to be operably coupled to an air duct hose, wherein the air duct hose atmospherically couples the negative air discharge apparatus with the negative air machine;
a plurality of air filters, said plurality of air filters being disposed in the interior volume of said housing; and
an air discharge port, said air discharge port being proximate said lower end of housing, said air discharge port configured to direct air outwards from said housing that has entered thereinto via said air intake.

8. The negative air discharge apparatus as recited in claim 7, and further including a second access, said second access being formed in one of said plurality of walls of said housing, said second access having an opening, said second access providing entry into the interior volume of said housing via said opening thereof, said second access having a door, said door of said second access being movable intermediate a closed position and an open position.

9. The negative air discharge apparatus as recited in claim 8, and further including an air deflector, said air deflector configured to provide directional control of airflow exiting said air discharge port, said air deflector being proximate the bottom of said housing.

10. The negative air discharge apparatus as recited in claim 9, wherein the plurality of walls of said housing has antimicrobial material disposed on the outer surface thereof.

11. The negative air discharge apparatus as recited in claim 10, and further including a pressure measuring unit, said pressure measuring unit operably coupled to said housing, said pressure measuring unit configured to measure and display the air pressure within the housing of the negative air discharge apparatus.
